(19) United States
(12) Patent Application Publication
(10) Pub. No.: US 2019/0049420 A1
(43) Pub. Date: Feb. 14, 2019

(54) INSPECTION SYSTEM AND INSPECTION APPARATUS

(71) Applicant: KYOCERA Corporation, Kyoto (JP)

(72) Inventor: Hirohide YAMASAKI, Yokohama·shi, Kanagawa (JP)

(73) Assignee: KYOCERA Corporation, Kyoto (JP)

(21) Appl. No.: 16/073,371

(22) PCT Filed: Jan. 16, 2017
(86) PCT No.: PCT/JP2017/001249

§ 371 Date: Jul. 27, 2018

(30) Foreign Application Priority Data
Jan. 29, 2016 (JP) 2016-015368

(51) Int. Cl. G01N 33/02; F25D 17/04; A61L 9/04

(52) U.S. Cl. CPC G01N 33/02; F25D 2317/0415; A61L 9/04; F25D 17/042

(57) ABSTRACT

An inspection system includes a sealable container and a detection apparatus that detects, in accordance with an inspection operation, a substance emitted by a specimen. When the specimen and the detection apparatus are stored in the container and the inspection operation is performed, the detection apparatus stored in the container detects the substance emitted by the specimen stored in the container.

### CROSS-REFERENCE TO RELATED APPLICATION

The present application claims priority to and the benefit of Japanese Patent Application No. 2016-015368 filed Jan. 29, 2016, the entire contents of which are incorporated herein by reference.

### TECHNICAL FIELD

The present disclosure relates to an inspection system and an inspection apparatus.

### BACKGROUND

A food product management system for managing the deterioration status of a food product is known. For example, patent literature (PTL) 1 discloses a food product management system that collects information related to the deterioration status of a food product stored in a container and manages the deterioration status.

### CITATION LIST

### Patent Literature

PTL 1: JP2005173675A

### SUMMARY

An inspection system according to an embodiment includes a sealable container and a detection apparatus that detects, in accordance with an inspection operation, a substance emitted by a specimen. When the specimen and the detection apparatus are stored in the container and the inspection operation is performed in the inspection system, the detection apparatus stored in the container detects the substance emitted by the specimen stored in the container.

An inspection apparatus according to an embodiment includes a detector and a communication interface. In accordance with an inspection operation, the detector detects a substance emitted by a specimen. The communication interface transmits information related to the substance emitted by the specimen and detected by the detector to an inspection apparatus configured to judge a quality of the specimen on the basis of the information related to the substance.

An inspection apparatus according to another embodiment includes a detector, a memory, and a controller. In accordance with an inspection operation, the detector detects a substance emitted by a specimen. The memory stores information associating the substance emitted by the specimen and a quality of the specimen. The controller refers to the information stored in the memory to judge the quality of the specimen on the basis of the substance detected by the detector.

### BRIEF DESCRIPTION OF THE DRAWINGS

In the accompanying drawings:

FIG. 1 is an outline illustrating an example of a form of using an inspection system according to a first embodiment;

FIG. 2 is a functional block diagram illustrating the schematic configuration of the inspection system in FIG. 1;

FIG. 3 is a sequence diagram illustrating an example of an inspection process executed by the inspection system of FIG. 1; and

FIG. 4 is a functional block diagram illustrating the schematic configuration of an inspection apparatus according to a second embodiment.

### DETAILED DESCRIPTION

The food product management system disclosed in PTL 1 continuously monitors the deterioration status of the food product stored in the container. Therefore, when the user of the food product management system disclosed in PTL 1 wishes to learn the deterioration status of a food product not stored in the container, for example, he cannot do so immediately. The present disclosure can improve the usefulness of an inspection system and an inspection apparatus.

Embodiments are described below in detail with reference to the drawings.

### First Embodiment

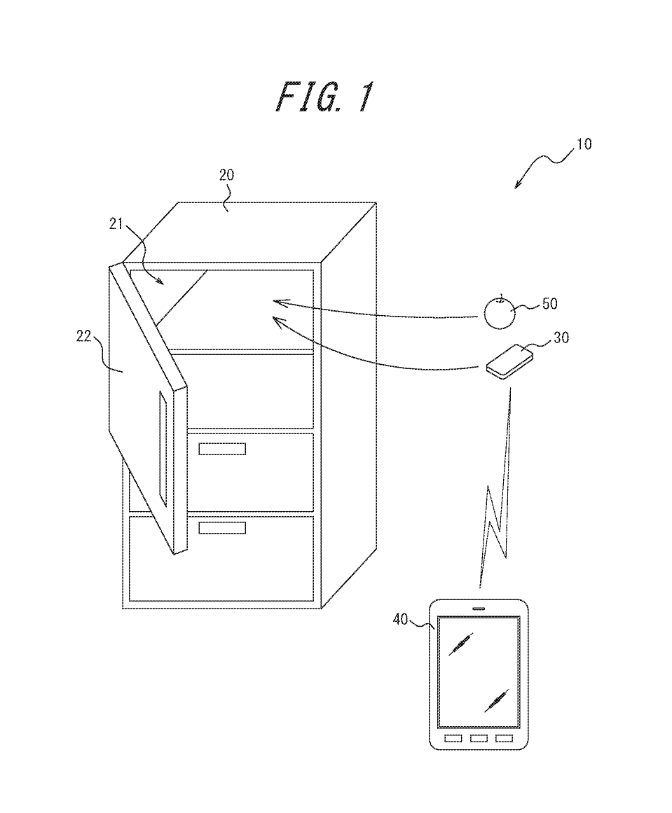

FIG. 1 is an outline illustrating an example of a form of using an inspection system according to the first embodiment. The inspection system 10 includes a container 20, a detection apparatus 30, and an inspection apparatus 40. The user of the inspection system 10 stores the detection apparatus 30 and a specimen 50 in the container 20, and with the container 20 sealed, the user detects a substance emitted by the specimen 50 using the detection apparatus 30. The detection apparatus 30 and the inspection apparatus 40 are communicably connected by short range wireless technology, such as Bluetooth® or Wi-Fi® (Bluetooth and Wi-Fi are each a registered trademark in Japan, other countries, or both). In the present embodiment, an example of the specimen 50 being a food product is described below.

The container 20 is configured to be hermetically sealable. The container 20 includes a containment chamber 21 in which the detection apparatus 30 and a food product (specimen 50) are removably stored. In the example in FIG. 1, a shelf is provided in the containment chamber 21 of the container 20, and the user can place the detection apparatus 30 and the food product on the shelf. The user opens a pivotable door 22 of the container 20 and stores the detection apparatus 30 and a food product in the containment chamber 21. The door 22 need not be pivotable. It suffices for the door 22 to adopt a state allowing the user to store the detection apparatus 30 and a food product in the containment chamber 21. For example, the door 22 may be slidable. An example of the container 20 being a refrigerator is described below in the present embodiment.

The detection apparatus 30 detects a substance emitted by the food product that is the specimen 50. The detection apparatus 30 may, for example, be provided with an odor sensor and detect an odor emitted by the food product.

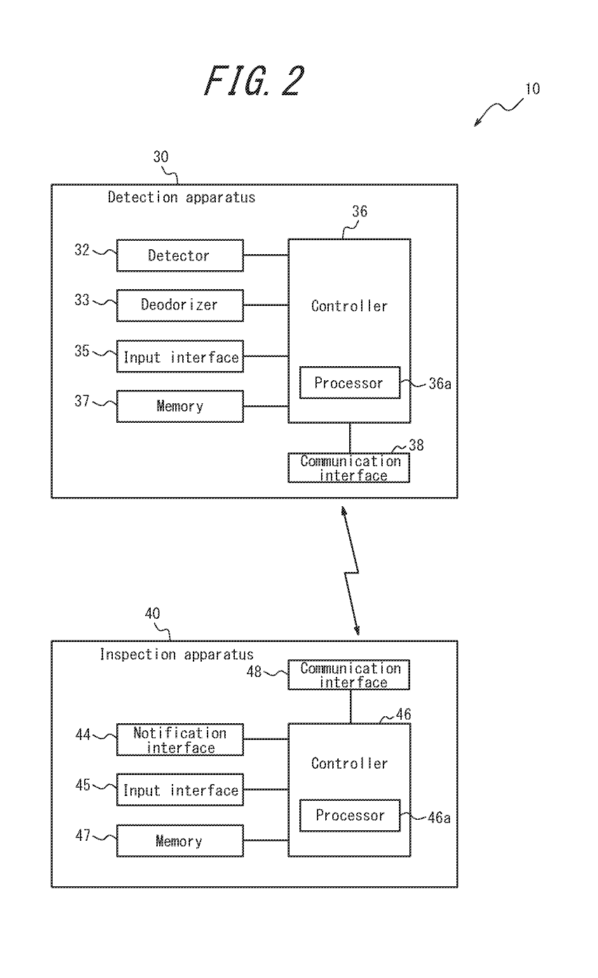

FIG. 2 is a functional block diagram illustrating the schematic configuration of the inspection system 10 in FIG. 1. As illustrated in FIG. 2, the detection apparatus 30 includes a detector 32, a deodorizer 33, an input interface 35, a controller 36, a memory 37, and a communication interface 38.

The detector 32 detects a substance emitted by the food product that is the specimen 50 in the container 20. The detector 32 may, for example, be an odor sensor that detects an odor emitted by the food product. The detector 32 may, for example, be a gas sensor that detects a gas emitted by the food product. Specifically, the detector 32 includes a sensitive membrane and a transducer. The sensitive membrane adsorbs gas molecules that are the source of the odor, and the transducer converts detection of the gas molecules in the sensitive membrane into an electric signal.

The detector 32 may, for example, be provided with a quartz crystal resonator type odor sensor that includes a sensitive membrane, made of an organic thin film, and a quartz crystal resonator. The quartz crystal resonator type odor sensor detects an odor by a change in the resonance frequency of the quartz crystal resonator when gas molecules are adsorbed on the sensitive membrane. The quartz crystal resonator functions as a transducer that converts detection of gas molecules into an electric signal.

The detector 32 may, for example, be provided with a semiconductor gas sensor. The semiconductor gas sensor detects the gas concentration by a change in the resistance of a metal oxide semiconductor when gas molecules are adsorbed on the metal oxide semiconductor. The oxide semiconductor functions as a transducer that converts detection of gas molecules into an electric signal. The detector 32 may, for example, be provided with an infrared absorption spectrum type gas sensor, an electrochemical gas sensor, a contact combustion type gas sensor, a biosensor, or the like.

To adsorb different types of gas molecules, for example, the detector 32 may be provided with a plurality of types of sensitive membranes or metal oxides. The detector 32 may be provided with a plurality of quartz crystal resonator type odor sensors, a plurality of semiconductor gas sensors, or a combination of these sensors. The detector 32 transmits the electric signal converted by the transducer to the controller 36 as odor-related information.

The deodorizer 33 performs a deodorization process inside the containment chamber 21. For example, the deodorizer 33 may be configured by a deodorizing apparatus. When the deodorizer 33 is configured to include an ozone deodorizing apparatus, the deodorizer 33 discharges ozone into the containment chamber 21. The discharged ozone decomposes gas molecules to deodorize the inside of the containment chamber 21. When the deodorizer 33 is configured to include an oxygen cluster deodorizing apparatus, the deodorizer 33 ionizes the oxygen molecules inside the containment chamber 21. The ionized oxygen molecules (oxygen cluster ions) subject gas molecules inside the containment chamber 21 to ion decomposition to deodorize the inside of the containment chamber 21. The detection apparatus 30 can deodorize the inside of the containment chamber 21 by performing the deodorization process using the deodorizer 33.

The input interface 35 accepts operation input from the user and may be configured by operation buttons (operation keys), for example. In the present embodiment, the input interface 35 may, for example, be a power button for turning on the detection apparatus 30. The input interface 35 may, for example, be an inspection operation button for causing the detection apparatus 30 to start the inspection process. For example, in accordance with an inspection operation on the input interface 35, the detection apparatus 30 detects an odor emitted by the food product stored in the containment chamber 21.

The controller 36 is a processor that controls and manages the detection apparatus 30 overall, starting with the functional blocks of the detection apparatus 30. The controller 36 is a processor, such as a central processing unit (CPU), that executes a program prescribing control procedures. Such a program is, for example, stored in the memory 37 or on an external storage medium.

To provide control and processing capability for executing various functions, as described below in greater detail, the detection apparatus 30 is provided with the controller 36, which includes one or more processors 36a.

In various embodiments, the one or more processors 36a may be implemented as a single integrated circuit or as a plurality of communicatively connected integrated circuits and/or discrete circuits. The one or more processors 36a can be implemented with a variety of known techniques.

In an embodiment, the processor 36a includes one or more circuits or units configured to execute one or more data calculation procedures or processes by executing instructions stored in related memory, for example. In another embodiment, the processor 36a may be firmware (such as discrete logic components) configured to execute one or more data calculation procedures or processes.

In various embodiments, the processor 36a may include one or more processors, controllers, microprocessors, microcontrollers, application specific integrated circuits (ASIC), digital signal processors, programmable logic devices, field programmable gate arrays, any combination of these devices or structures, or a combination of other known devices and structures, to execute the below-described functions.

The controller 36 executes the odor detection process using the detection apparatus 30 by controlling the entire detection apparatus 30. For example, the controller 36 activates the detector 32 in accordance with an inspection operation from an external source (inspection apparatus 40). The detector 32 activated by the controller 36 starts to detect an odor emitted by the food product. The controller 36 acquires information related to the odor detected by the detector 32 from the detector 32.

The controller 36 wirelessly transmits the odor-related information detected by the detector 32 to the inspection apparatus 40 through the communication interface 38.

The memory 37 may be configured with a semiconductor memory, a magnetic memory, or the like. The memory 37 stores a variety of information, programs for causing the detection apparatus 30 to operate, and the like and also functions as a working memory. The memory 37 may, for example, store the result of the odor detection process by the detection apparatus 30 temporarily (for a predetermined time period).

The communication interface 38 exchanges a variety of information by communicating with the inspection apparatus 40. For example, the communication interface 38 transmits the odor-related information to the inspection apparatus 40. The odor-related information may, for example, be transmitted from the detection apparatus 30 to the inspection apparatus 40 each time the controller 36 acquires the odor-related information from the detector 32. The odor-related information may, for example, be transmitted from the detection apparatus 30 to the inspection apparatus 40 when the user provides predetermined operation input to the detection apparatus 30.

The inspection apparatus 40 may, for example, be configured as a terminal. The inspection apparatus 40 includes a notification interface 44, an input interface 45, a controller 46, a memory 47, and a communication interface 48.

The notification interface 44 provides notification of the result of the inspection process by the inspection apparatus 40. The notification interface 44 can, for example, provide notification by a visual method using image, character, or color display, light emission, or the like; an auditory method using audio or the like; or a combination of these methods. To provide notification with a visual method, the notification interface 44 may be a display device that provides notification by displaying images or characters. The notification interface 44 may, for example, provide notification by emitting light with an LED or the like. To provide notification with an auditory method, the notification interface 44 may be a speaker or other such sound generating device that provides notification by outputting an alarm, audio guidance, or the like. The notification interface 44 is not limited to providing notification by a visual or auditory method. Any method recognizable by the subject may be adopted. For example, the notification interface 44 may provide notification with a vibration pattern or the like.

The input interface 45 accepts operation input from the user and may be configured by operation buttons (operation keys), for example. The input interface 45 may be configured by a touchscreen and accept touch operation input from the user to an input region, displayed on a portion of the display device that is the notification interface 44, for accepting operation input. For example, in accordance with an inspection operation on the input interface 45, the detection apparatus 30 detects an odor emitted by the food product stored in the containment chamber 21.

The controller 46 controls and manages the inspection apparatus 40 overall, starting with the functional blocks of the inspection apparatus 40. To provide control and processing capability for executing various functions, the controller 46 may include at least one processor 46a. The controller 46 is a processor, such as a CPU, that executes a program prescribing control procedures. Such a program is, for example, stored in the memory 47 or on an external storage medium.

The controller 46 judges the quality of the food product on the basis of the odor-related information received by the communication interface 48 from the detection apparatus 30. In other words, the controller 46 judges the quality of the food product on the basis of the odor-related information acquired by the detection apparatus 30 from the detector 32. For example, the detector 32 may include a plurality of different odor sensors that adsorb ethylene, ammonia, alcohol, aldehyde, and sulfur gas molecules. The controller 46 may judge the quality of the food product on the basis of a plurality of signals detected by the plurality of odor sensors. The components of gas molecules that are the source of the odor emitted by a food product, the ratio of the components, and the like differ depending on the food product and the quality of the food product. Therefore, the controller 46 judges the quality of a food product on the basis of odor-related information, i.e. the components of gas molecules that are the source of the odor, the ratio of the components, and the like. The controller 46 may, for example, judge the quality of the food product on the basis of the outputs of a plurality of odor sensors and the ratio of the outputs. The controller 46 may, for example, judge the quality of the food product on the basis of features (output value, time constant, or the like) of the response of a plurality of odor sensors. The detector 32 may, for example, include a plurality of ethylene, alcohol, sulfur, and ammonia type odor sensors. When judging the quality of a vegetable, for example, the controller 46 may judge that the quality has deteriorated when the outputs of the alcohol, sulfur, and ammonia type odor sensors among the plurality of odor sensor outputs increase to a predetermined value or higher. The controller 46 refers to information (data), stored in the memory 47, associating the odor emitted by a food product and the quality of the food product to judge the quality of the food product. Here, the quality of a food product refers to the properties or qualities of the food product.

The quality of the food product may, for example, refer to whether the food product is spoiled. In this case, the controller 46 judges whether the food product is spoiled on the basis of the odor-related information. The controller 46 may judge the degree of spoiling of the food product.

The quality of the food product may, for example, refer to whether the food product is ripe. In this case, the controller 46 judges whether the food product is ripe on the basis of the odor-related information. The controller 46 may judge the time at which the food product will be ripe.

The quality of the food product may, for example, refer to the degree of aging of the food product. In this case, the controller 46 judges the degree of aging of a food product, i.e. the degree to which the food product (such as meat) has aged, on the basis of the odor-related information.

The quality of the food product may, for example, refer to the freshness of the food product. In this case, the controller 46 judges the freshness of a food product, i.e. how fresh the food product is, on the basis of the odor-related information.

The quality of the food product may, for example, refer to the production area of the food product. In this case, the controller 46 judges the production area of the food product on the basis of the odor-related information. Specifically, the controller 46 judges whether the food product is domestic or foreign, for example. The controller 46 may judge the specific production area of the food product.

The quality of the food product may, for example, refer to the extent of residual pesticide in the food product. In this case, the controller 46 judges the extent of residual pesticide in the food product, i.e. the amount of residual pesticides in the food product, on the basis of the odor-related information.

The quality of the food product is not limited to the above-described examples and may be any other property or quality related to the food product. The controller 46 may judge the odor using a statistical method, such as principal component analysis, or using a neural network. The controller 46 may generate data by performing a learning process in advance to extract the features of the response of a plurality of odor sensors for each quality of a food product. The controller 46 may store the data after learning in the memory 47. The controller 46 may then judge the quality of the food product on the basis of the degree of matching between the data after learning stored in the memory 47 and the data detected by the detector 32. Based on newly detected data, the controller 46 may update the data after learning stored in the memory 17.

The controller 46 provides notification of information related to the judged quality of the food product through the notification interface 44 as the result of the inspection process. As described above, the notification method may be any method recognizable by the subject. When the notification interface 44 is a display device, the controller 46 provides notification by display of images, characters, or colors on the display device.

The controller 46 stores information related to the judged quality of the food product in the memory 47 as history data, for example.

The memory 47 may be configured with a semiconductor memory, a magnetic memory, or the like. The memory 47 stores a variety of information, programs for causing the inspection apparatus 40 to operate, and the like and also functions as a working memory. For example, the memory 47 stores information (data) associating the odor emitted by a food product and the quality of the food product. The memory 47 may, for example, store the result of the detection process by the inspection apparatus 40 as history data.

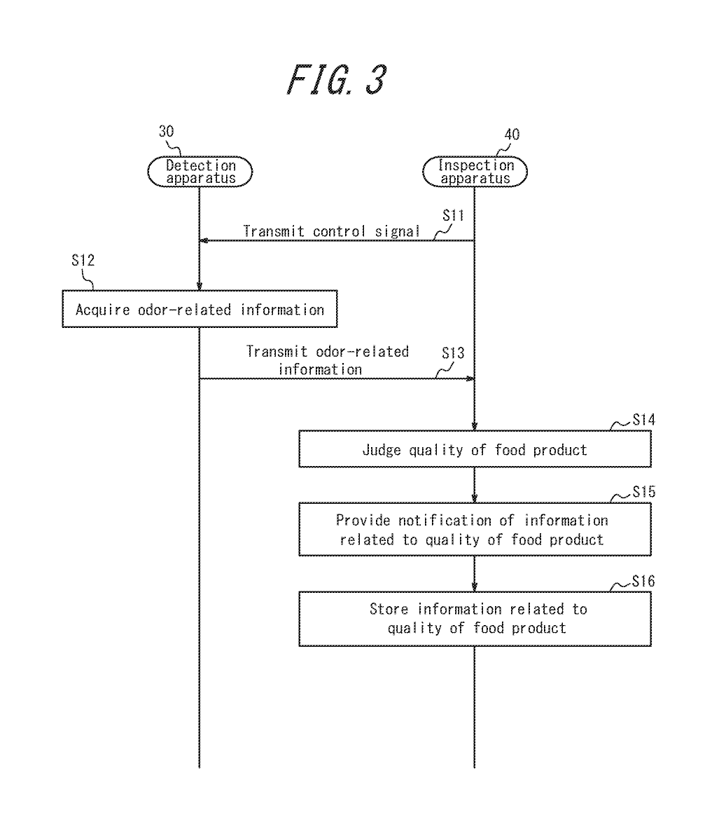

Next, an example of an inspection process by the inspection system 10 is described with reference to the sequence diagram in FIG. 3.

To execute the inspection process with the inspection system 10, the user first turns on the power to the detection apparatus 30. The user then stores the detection apparatus 30 and a food product in the containment chamber 21.

The user uses the input interface 45 of the inspection apparatus 40 to input an operation (inspection operation) for the detection apparatus 30 to execute the detection process. On the basis of user input of the inspection operation, the inspection apparatus 40 transmits a control signal to the detection apparatus 30 to execute the detection process (step S11). The user may input the inspection operation using the input interface 35 of the detection apparatus 30. In this case, the user inputs the inspection operation on the detection apparatus 30 before storing the detection apparatus 30 in the containment chamber 21.

The detection apparatus 30 then uses the detector 32 to acquire information related to an odor in the container 20 on the basis of a control signal acquired either from the inspection apparatus 40 or from the detection apparatus 30 itself (step S12).

The detection apparatus 30 transmits the acquired odor-related information to the inspection apparatus 40 (step S13).

On the basis of the odor-related information acquired from the detection apparatus 30, the inspection apparatus 40 refers to the data stored in the memory 47 to judge the quality of the food product (step S14).

The inspection apparatus 40 provides notification of information related to the quality of the food product judged in step S14 through the notification interface 44 (step S15). The user can learn the quality of the food product by the notification from the notification interface 44.

The inspection apparatus 40 stores the information related to the quality of the food product judged in step S14 in the memory 47 (step S16).

In this way, the detection apparatus 30 in the inspection system 10 according to the present embodiment detects an odor emitted by a food product stored in the containment chamber 21 while the detection apparatus 30 and the food product are stored in the containment chamber 21. On the basis of the odor detected by the detection apparatus 30, the inspection apparatus 40 judges the quality of the food product. Therefore, when the user wishes to learn the quality of a food product, the user can do so immediately by placing the food product and the detection apparatus 30 in the containment chamber 21 and causing the inspection system 10 to execute the inspection process. Accordingly, the inspection system 10 allows the quality of a food product to be inspected in accordance with user need, thereby improving usefulness as compared to known apparatuses.

The inspection system 10 according to the present embodiment allows one inspection system 10 to inspect the quality of a plurality of food products by switching of the food product stored in the containment chamber 21. When the food product stored in the containment chamber 21 is switched, the detection apparatus 30 may perform a deodorization process using the deodorizer 33.

Furthermore, the inspection system 10 according to the present embodiment can inspect the quality of a food product by the food product and the detection apparatus 30 being stored in any sealable container 20. Therefore, the inspection system 10 can inspect the quality of a food product anywhere the sealable container 20 is available.

In the inspection system 10 according to the above embodiment, the inspection apparatus 40 judges the quality of the food product. However, the quality of the food product need not be judged by the inspection apparatus 40 capable of communicating with the detection apparatus 30. The quality of the food product may, for example, be judged by a single inspection apparatus having the functions of both the detection apparatus 30 and the inspection apparatus 40 in the above embodiment. An example of the configuration of the inspection apparatus in this case is described below as a second embodiment.

### Second Embodiment

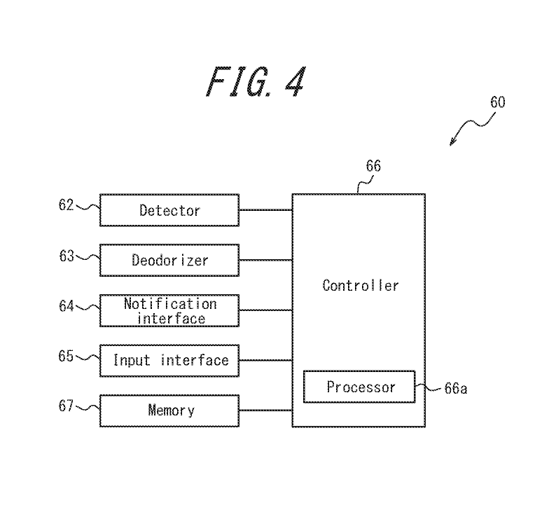

FIG. 4 is a functional block diagram illustrating the schematic configuration of an inspection apparatus according to the second embodiment. As illustrated in FIG. 4, an inspection apparatus 60 that both acquires the odor-related information and judges the quality of a food product includes a detector 62, a deodorizer 63, a notification interface 64, an input interface 65, and a controller 66.

Since the functions of the detector 62 and the deodorizer 63 are the same as those of the detector 32 and deodorizer 33 of the detection apparatus 30 described in the above embodiment, a description thereof is omitted. Since the functions of the notification interface 64 and the input interface 65 are the same as those of the notification interface 44 and the input interface 45 of the inspection apparatus 40 described in the above embodiment, a description thereof is omitted.

The controller 66 controls and manages the inspection apparatus 60 overall, starting with the functional blocks of the inspection apparatus 60. To provide control and processing capability for executing various functions, the controller 66 may include at least one processor 66a. The controller 66 controls the odor detection process by the detector 62. The controller 66 judges the quality of a food product on the basis of the odor-related information acquired from the detector 62. At this time, the controller 66 refers to information (data), for example stored in the memory 67, associating the odor emitted by a food product and the quality of the food product to judge the quality of the food product.

The controller 66 provides notification of information related to the judged quality of the food product through the notification interface 64. The controller 66 also stores information related to the judged quality of the food product in the memory 67 as history data, for example.

The memory 67 may be configured with a semiconductor memory, a magnetic memory, or the like. The memory 67 stores a variety of information, programs for causing the inspection apparatus 60 to operate, and the like and also functions as a working memory. For example, the memory 67 stores information (data) associating the odor emitted by a food product and the quality of the food product. The memory 67 may, for example, store the result of the detection process by the inspection apparatus 60 as history data.

When using the inspection apparatus 60 to execute the process to inspect the quality of a food product, the user first turns on the power to the inspection apparatus 60 for the inspection apparatus 60 to be capable of executing the detection process. In accordance with an inspection operation input to the input interface 65, the inspection apparatus 60 becomes capable of executing the detection process. The user then stores the inspection apparatus 60 and a food product in the container 20 and seals the container 20. The inspection apparatus 60 acquires information related to an odor in the containment chamber 21 and judges the quality of a food product on the basis of the acquired odor-related information. The inspection apparatus 60 provides notification of information related to the judged quality of the food product through the notification interface 64. The user can immediately learn the quality of the food product by removing the inspection apparatus 60 from the containment chamber 21 and confirming the notification from the notification interface 64.

The above-described inspection apparatus 60 can judge the quality of a food product on the basis of the odor-related information detected by the detector 62, without communicating with another apparatus.

The inspection system and inspection apparatus are not limited to the above embodiments, and a variety of modifications and changes may be made. For example, the functions and the like included in the components, steps, and the like may be reordered in any logically consistent way. Furthermore, components, steps, and the like may be combined into one or divided.

For example, the detection apparatus 30 has been described in the first embodiment as being an odor sensor that detects an odor emitted by a food product. The detection apparatus 30 may, however, be a sensor other than an odor sensor. For example, the detection apparatus 30 may be a gas sensor that detects odorless gas molecules emitted by the specimen.

For example, the detection apparatus 30 in the first embodiment need not include the deodorizer 33.

The inspection apparatus 40 in the first embodiment has been described as being configured as a terminal. However, the inspection apparatus 40 may be configured as a server, for example.

Bluetooth and Wi-Fi were listed as examples of means for the detection apparatus 30 and the inspection apparatus 40 to communicate in the first embodiment, but the means for communication are not limited to these examples. The communication between the detection apparatus 30 and the inspection apparatus 40 may be established by any other wireless communication means. The communication between the detection apparatus 30 and the inspection apparatus 40 may be established by a wired communication means. For example, in this case the user may store the detection apparatus 30 in the containment chamber 21 and have the detection apparatus 30 acquire information related to the odor in the containment chamber 21. The user may then remove the detection apparatus 30 from the containment chamber 21 and connect the detection apparatus 30 to the inspection apparatus 40 with a wire to transmit the odor-related information to the inspection apparatus 40.

### REFERENCE SIGNS LIST

10 Inspection system
20 Container
21 Containment chamber
22 Door
30 Detection apparatus
32, 62 Detector
33, 63 Deodorizer
35, 45, 65 Input interface
36, 46, 66 Controller
36a, 46a, 66a Processor
37, 47, 67 Memory
38, 48 Communication interface
40, 60 Inspection apparatus
44, 64 Notification interface
50 Specimen

1. An inspection system comprising:
a sealable container; and
a detection apparatus configured to detect, in accordance with an inspection operation, a substance emitted by a specimen; wherein
when the specimen and the detection apparatus are stored in the container and the inspection operation is performed, the detection apparatus stored in the container detects the substance emitted by the specimen stored in the container.

2. The inspection system of claim 1, wherein the container is a refrigerator.

3. The inspection system of claim 1, wherein the detection apparatus is configured to execute a deodorization process in the container.

4. The inspection system of claim 1, further comprising:
an inspection apparatus; wherein
the detection apparatus is configured to transmit information related to the detected substance to the inspection apparatus; and
the inspection apparatus is configured to receive the information related to the substance from the detection apparatus and to judge a quality of the specimen on the basis of the received information related to the substance.

5. The inspection system of claim 4, wherein the inspection apparatus comprises a memory configured to store information associating a substance emitted by a specimen and a quality of the specimen, and the inspection apparatus refers to the information stored in the memory to judge the quality of the specimen stored in the container.

6. The inspection system of claim 4, wherein the inspection apparatus comprises a notification interface configured to provide notification of information related to the judged quality.

7. The inspection system of claim 4, wherein
the specimen is a food product; and
as the quality of the food product, the inspection apparatus is configured to judge at least one of whether the food product is spoiled, whether the food product is ripe, a degree of aging of the food product, a freshness of the food product, a production area of the food product, and an extent of residual pesticide in the food product.

8. The inspection system of claim 1, wherein the detection apparatus is configured to judge a quality of the specimen on the basis of information related to the detected substance.

9. An inspection apparatus comprising:
a detector configured to detect, in accordance with an inspection operation, a substance emitted by a specimen; and
a communication interface configured to transmit information related to the substance emitted by the specimen and detected by the detector to an inspection apparatus configured to judge a quality of the specimen on the basis of the information related to the substance.

10. An inspection apparatus comprising:
a detector configured to detect, in accordance with an inspection operation, a substance emitted by a specimen;
a memory configured to store information associating a substance emitted by the specimen and a quality of the specimen; and
a controller configured to refer to the information stored in the memory to judge the quality of the specimen on the basis of the substance detected by the detector.
